(19) United States
(12) Patent Application Publication
(10) Pub. No.: US 2005/0102003 A1
(43) Pub. Date: May 12, 2005

(54) Perficardial pacing lead placement device and method

(76) Inventors: James R. Grabek, Minneapolis, MN (US); Michael Hoey, Shoreview, MN (US)

Correspondence Address: Beck & Tysver, P.L.L.C., Suite 100, 2900 Thomas Avenue S., Minneapolis, MN, 55416, US

(21) Appl. No.: 10/691,815

(22) Filed: Oct. 23, 2003

Related U.S. Application Data
(63) Continuation-in-part of application No. 09/565,059, filed on May 3, 2000.
(60) Provisional application No. 60/421,541, filed on Oct. 25, 2002.

(51) Int. Cl. A61N 1/362

(52) U.S. Cl. 607/27

(57) ABSTRACT

The present invention relates to a method and apparatus for pacing the ventricle of a patient's heart through the pericardial space, including an epicardial lead and a method for placement of the lead.

### CROSS REFERENCE TO RELATED APPLICATION

The present application is a Continuation-In-Part of U.S. patent application Ser. No. 09/565,059 filed May 3, 2000 which is co-pending with the present application, and which is incorporated by reference in its entirety. The present application claims the benefit of provisional application 60/421,541 filed Oct. 25, 2002 which is incorporated by reference in its entirety.

### BACKGROUND OF THE INVENTION

The earliest pacemakers relied on an implanted lead system which was coupled to the epicardial surface of the heart. The electrodes were typically in the form of exposed metal coils which were sutured into stab wounds made on the epicardial surface of the ventricle. Surgical access to this site was through the chest and required breaking the sternum.

Pacing as a therapy was adopted more widely with the introduction of a transvenous lead system which permitted the physician to place stimulation electrodes in the heart without the necessity for thoracic surgery. Although transvenous leads are widely accepted for both pacing and defibrillation therapy, they still possess shortcomings which cannot be readily addressed by vascular access leads.

For example, bi-ventricular pacing requires that specific areas of the ventricle be stimulated, and these are not reliably accessed with a transvenous approach, nor can they be reached with conventional thoracic surgery.

### SUMMARY OF THE INVENTION

In contrast to the prior art, the present invention teaches both methods and devices for placing leads on the surface of the heart within the pericardial space.

Access to the pericardial space may be made through any one of a number of techniques, most preferably through the use of a Perducer device available from Comedicus of Minneapolis Minn. The epicardial lead contains design features which permit it to be readily affixed to the epicardial surface of the heart. Such as barbs or other fixation devices that revealed or deployed through the use of a stylet which is manipulated through the body of the pericardial access device. A scope or other visualization techniques may be exercised while placing the lead to ensure that anatomical reference points are detected and that lead placement is proper.

### BRIEF DESCRIPTION OF DRAWINGS

Identical reference numerals describe identical structure in the figures wherein:

FIG. 1 is a schematic diagram showing the use of a multiple electrode lead;

FIG. 2 is a schematic diagram of a pacing lead.

### DETAILED DESCRIPTION

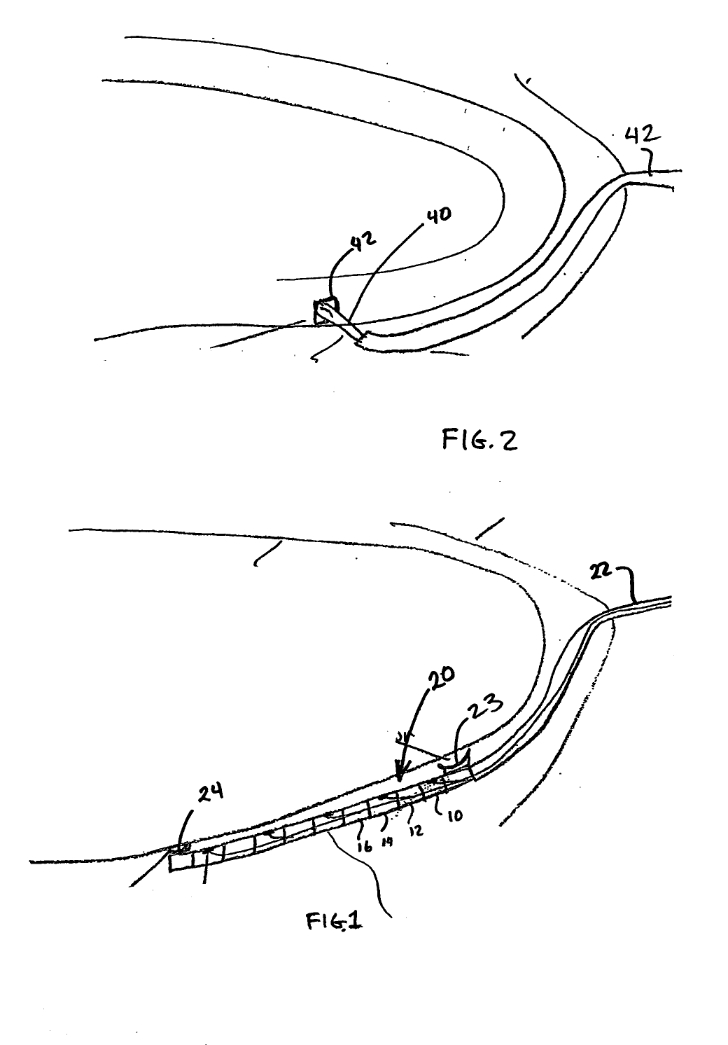

FIG. 1 shows a multiple link temporary pacing lead with electrode segments such as 10 and 14 separated by articulated insulators such as 12 and 16. Each electrode can be used to test the pacing parameters at the location associated with the electrode. It is desirable to determine the optimal pacing site especially for biventricular pacing. The articulated catheter 20 can be delivered to the posterior side of the heart and the right and left ventricle can each be accessed and paced. The distal anchor 24 can be a mechanical barb or a suction device to temporally fix the lead into position while the heart is tested. The lead may be stabilized in place with an optional proximal anchor 23 as well.

FIG. 2 shows a pacing lead with a barbed anchor that can be rotated in to attachment to the heart surface after placement on the posterior surface of the heart.

Taken as a system the catheter 20 of FIG. 1 can be used to explore and select the optimal pacing site. The pericardial access allows this site to be found on the right and left heart on the posterior surface. The lateral barbs on the pacing lead allow the lead 40 to be affixed to the posterior heart surface if required.

1. A method of selecting a pacing site on the heart comprising:
entering the pericardial space with a pacing lead having an asymmetrical pacing electrode;
manipulating the lead inside the intact pericardial sack while taking pacing thresholds at multiple location on the heart;
selecting and attaching the lead at one of said locations without removing the pericardium.
